(19) United States
(12) Patent Application Publication
(10) Pub. No.: US 2002/0164659 A1
(43) Pub. Date: Nov. 7, 2002

(54) Analytical methods and compositions

(76) Inventors: Galla Chandra Rao, Princeton, NJ (US); Leon Wmm Terstapper, Huntingdon Valley, PA (US)

Correspondence Address: JAMES WILCOX, IMMUNICON CORP, SUITE 100, 3401 MASONS MILL ROAD, HUNTINGDON VALLEY, PA, 19006, US

(21) Appl. No.: 10/017,437

(22) Filed: Dec. 18, 2001

Related U.S. Application Data
(60) Provisional application No. 60/253,985, filed on Nov. 30, 2000.

(51) Int. Cl. C12Q 1/68; G01N 33/53

(52) U.S. Cl. 435/7.5; 435/6

(57) ABSTRACT

Compositions and analytical methods for detection of a single target entity or for simultaneous detection of multiple target entities by means of non-magnetic microparticles operably linked with a first binding partner capable of binding to a complementary second binding partner which, in turn, is operably linked to smaller magnetic particles to form a layer of magnetic particles on the non-magnetic microparticles.

### CROSS REFERENCE TO RELATED APPLICATIONS

This application claims priority under 35 U.S.C. section 119 (e) to U.S. Provisional Application No. 06/253,985, filed Nov. 30, 2000, the entire disclosure of which is incorporated by reference herein.

### BACKGROUND OF THE INVENTION

This invention relates generally to analytical methods and compositions, specifically, to the preparation and use of compositions consisting of biotinylated unlabeled or detectably labeled microparticles, streptavidin-coated magnetic particles, biotinylated binding partners, and detectably labeled binding partners to form labeled magnetic complexes bearing one or more different labels which complexes, when premixed as cocktails, are useful in the simultaneous isolation and analysis of one or more target entities in sample specimens.

### BACKGROUND ART

1. Non-magnetic Separations

Inorganic and organic microparticles, herein defined as particles in the size range of 0.5 to 30 um, have numerous applications in solid phase separations ranging from immunoassays and hybridization assays to cell separations. Most commonly used are polymer microparticles made of polystyrene also known as latex beads which are commercially available in different sizes (0.5 to 20 um), some with essentially monodisperse size distributions. Inorganic microparticles of controlled sizes are also usable for these applications. Such microparticles or beads have been coupled to biotinylated binding partners including biotinylated antibodies, for specific or selective separation of soluble and particulate target entities, herein defined as analytes, and including, for example, antigens, peptides, proteins, carbohydrates, oligonucleotides (oligomers), cells, rare cells and tumor cells, from complex mixtures by means of centrifugation or filtration. For some analytical applications, the microparticles can be doped or coupled to a single or a plurality of fluorescent dyes and used as calibrators, e.g. in flow cytometric analysis of antigens, oligonucleotides and cells. Luminex Corp. (U.S. Pat. Nos. 5,763,330, 5,981,180, and U.S. Pat. No. 6,057,107) uses subsets of fluorescent multi-fluor microparticles or beads, bearing a plurality of fluorescent labels at different ratios, to simultaneously measure up to 100 analytes in a single specimen. However, the Luminex particles are not magnetic and do not allow magnetic enrichment of rare target entities present in very low concentrations. For example, non-magnetic microparticles require enrichment by centrifugation or density gradient centrifugation for selective separation of particulate species, for example in cell isolation, where captured target cells need to be separated from non-target cells of higher or lower densities before analysis. Automation of centrifigation steps can be done but complicates high throughput assay protocols.

2. Magnetic Separations

In contrast, separation and analysis of soluble analytes or particulate target materials from non-target materials by magnetic means is relatively easily done with magnetic or more appropriately with superparamagnetic particles, herein defined as magnetically responsive nanoparticles, which become reversibly magnetic and non-magnetic when placed inside or outside a magnetic field, respectively. Superparamagnetic particles may be divided on the basis of size or their colloidal properties. Superparamagnetic and magnetic particles (hereafter used interchangeably) of diverse sizes and compositions are widely available or disclosed in the art. Magnetic particles, below 0.2 um in size, settle very slowly by gravity and are designated as magnetic colloidal particles, ferrofluids or magnetic nanoparticles. The larger magnetic particles, larger than 0.5 um in size, settle faster and are also designated as magnetic microparticles. The applications in these inventions are confined to magnetic nanoparticles in the size range of 50 to 1000 nm. Magnetic particle compositions have been described in U.S. Pat. Nos. 3,970,518; 4,018,886; 4,230,685; 4,267,234; 4,452,773; 4,554,088 and 4,659,678.

3. Magnetic Separation Devices

Magnetic nanoparticles require high gradient magnetic separators (HGMS) with either externally or internally generated magnetic gradients for rapid and efficient separations. The larger magnetic microparticles (>0.5 um) can generally be separated with conventional external low gradient magnets, e.g. in so-called magnetic racks.

External HGMS separations are performed in magnetic fields generated external to the specimen container as in the patented quadrupole, hexapole or other configurations for separations in test tubes as described in U.S. Pat. Nos. 5,186,827; 5,200,084 and 5,466,574, assigned to Immunivest Corp. Even higher magnetic fields are obtained with internal HGMS, which are formed inside the specimen container in intimate contact of the test fluid with a paramagnetic material, such as magnetizable stainless steel wool. Miltenyi Inc. sells small magnetic nanoparticles in the 50 to 100 nm size range requiring internal HGMS systems. However, external HGMS, and particularly separation in magnetic racks, are preferred for simplicity in processing and high sample throughput. Hence, magnetic particles of 0.5 to 3 um in diameter coated with specific binding partners for target entities are preferred for separation of soluble analytes and particulate target entities in blood, body fluids, and other test media herein defined as specimen samples. Several vendors (e.g. Bang's Labs, Polysciences, Inc., Spherotech, Inc., Dynal) provide magnetic particles in this size range, some even bearing streptavidin or other binders suitable for selective magnetic separations. Wang et al. (U.S. Pat. No. 5,091,206) disclosed a process for producing magnetically responsive particles bearing a surface layer of magnetite on a polymeric core in which the magnetite is encapsulated. Hence, the magnetic properties of these particles are essentially fixed during manufacture and thus difficult to modulate for specific assay applications. Wang also does not disclose analytical methods for simultaneous analysis of a plurality of target entities.

4. Characteristics of Binding Partners

Briefly, binding partners have affinity for specific amino acid (defined as epitopes) or nucleotide sequences on the target entities. For example, in sandwich immunoassays, two types of binding partners or antibodies are used: one antibody for capture and a second detectably labeled antibody for quantitation. In hybridization assays, the capture binder consist of a nucleotide sequence or probe for capturing a segment on the target entity and the detection binder may be a detectably labeled complementary nucleotide sequence.

The capture binder is conventionally labeled with biotin to permit attachment to a solid phase, which is coated with an avidin species. The biotin-avidin or biotin-streptavidin interactions have very high affinities, about (10E+15) 1/M, thereby substantially enhancing the stability of the resultant complexes bearing the captured target entities.

The detection binding partner with affinity or complementarity for the target entity typically bears a fluorescent label permitting qualitative or quantitative analysis of the target entity.

5. Assay Optimization

Conventional magnetic particles are made mainly by incorporating the magnetite inside the particle during manufacture, thereby permitting varying the magnetite contents only during preparation of the magnetic particles. However, further modulation of the magnetic particles may be required to alter the binding characteristics of the microparticles to compensate for variations in specific epitope densities or sequencc complementarities on the target entities, which may vary substantially among different target entities.

Unlabeled microparticles may be used for the analysis of a single target entity in a sample specimen. However, for simultaneous analysis of multiple target entities in a single specimen sample, as exemplified by the Luminex system, the assays are performed with subsets of multiple types of non-magnetic particles each bearing a different detectable label or different ratios of such detectable labels. The methodologies of this invention substantially enhance the sensitivity and scope of the Luminex art by utilizing novel labeled magnetic particle compositions providing means for both capture and differentiation of target entities in combination with additional specific and non-specific binding partners detectably labeled with different labels, thereby providing further dicrimination of multiple target entities in a given specimen sample.

Thus the compositions of this invention permit optimization of the labeled binding partners for either detection a single target entity by means of a single label or of multiple target entities by means of multiple detectable labels as well as multiple label ratios.

6. Collection, Detection and Analysis

Detection and quantitation of magnetically selected soluble analytes and particulate target entities, labeled with appropriate detectable labels, is routinely done by flow cytometry using instruments manufactured by Becton Dickinson, Coulter Diagnostics and Ortho Clinical Diagnostics. Analytical complexes bearing target entities, for example antigens, oligonucleotides, peptides, proteins, and cells can be analyzed and enumerated by flow cytometry as detectable events on the basis of size, optical label type and optical label intensities. The methods disclosed in this invention not only permit simultaneous measurement of more than one target entity in complex specimen samples as practice in the art, but also facile magnetic enrichment of target entities present at very low concentrations prior to analysis.

7. Particle Optimization

Besides affecting particle densities, the magnetite loading of the microparticles also needs to be adjusted to provide optimal collection rates of target entities when placed in the magnetic field, i.e. collection should be complete in about 10 minutes, preferably in about 5 to 10 minutes at low particle densities. Magnetic fractionation of captured target entities or separation from free magnetic complexes likewise requires magnetic particles optimized for a specific magnetic separation system. Magnet systems that spread the collected magnetic particles over a wider area, e.g. the aforementioned quadrupole or hexapole HGMS, are preferred to minimize trapping of non-target species leading to nonspecific binding. While conventional magnetic particles are available in numerous sizes, different particle densities and different magnetic loading levels, it is difficult or at least tedious to optimize the collection parameters of the magnetic particles for a given magnet configuration.

A limited number of different non-magnetic and magnetic streptavidin-coated microparticles of variable sizes and bearing varying levels of streptavidin, are available from several vendors (for example, Bang's Lab, Polysciences, CPG Inc.). Conventional protocols for optimization of parameters such as streptavidin levels, particle size, particle densities, and magnetite content can be used to achieve a desired separation efficiency for a given separation, but obviously numerous permutations of these parameters need to be experimentally evaluated for specific applications. Review of the current art clearly establishes a need for improved and simpler optimization of magnetic collection materials, more efficient collection techniques and improved analytical methods for simultaneously analyzing multiple target entities present at low levels in specimen samples. The compositions and methods of this invention largely satisfy overcome the above-cited deficiencies and also allow simultaneous analysis of more than one target entity by means of different detectable labels and variable label ratios.

8. Detectable Labels

Detectable labels are primarily optical, and preferably, fluorescent, both organic and inorganic, which can be excited by means of lasers, laser diodes or filtered light sources. Multiple fluorescent labels are conventionally used in combination at variable ratios are selected so as not to substantially overlap in their emission spectra, as exemplified by the Luminex art. Numerous fluorescent materials are known in the art that can be incorporated into microparticles, for example, during emulsion polymerization as in the case of the Luminex particles.

These methods are also used in this invention in conjunction with novel labeling methods, which include specific labels attached to the magnetic particles either directly or by means of labeled binding partners, wherein such binding partners include biotin, streptavidin, proteins, antibodies and oligonucleotide sequences. Among the numerous available fluors or fluorophores described in the art, phycocyanines (for example, phycoerythrin, allophycocyanin) and cyanines, including the CY™ dyes, are particularly preferred as optically detectable labels used in this invention. The novel methods disclosed in the instant invention permit simultaneous differentiation or detection of up to about 50 different target entities in a single specimen by flow cytometry, which is comparable to the claimed performance of about 64 analytes for each particle subset in the Luminex methodologies. A greater number of permutations enabling detection of target entities can be achieved with the methodologies of this invention by varying the following parameters: size of microparticle; type, level and ratio of the labels inside the microparticles; type and level of the label attached to the magnetic particles; type and level of the label specifically bound to the captured target entities. The main limitation is not set by these parameters, but by the instrumental capabilities in resolving labeled target complexes as encountered, for instance, in flow cytometry instruments.

### SUMMARY OF THE INVENTION

This invention provides compositions and analytical methods for detection of a single target entity or for simultaneous detection of multiple target entities by means of non-magnetic microparticles operably linked with a first binding partner capable of binding to a complementary second binding partner which, in turn, is operably linked to smaller magnetic particles to form a layer of magnetic particles on the non-magnetic microparticles, wherein this layer of magnetic particles still possess free binding sites distal to the contact area between the magnetic particle and the microparticles. These free binding sites are available for binding at least three additional entities: 1. a third target specific binding partner which is operably linked to first binding partner, 2. a detectably labeled fourth binding partner or a label both operably bound to first binding partner, and 3. a blocking species with affinity for first binding partner. Additional labeled target specific fifth binding partners, recognizing available epitopes on bound target entities, provide further differentiation and discrimination of target entities. First binding partner is exemplified by a biotin species and second binding partner may be an avidin species. The third binding partner may be an antibody or olignucleotide probe and the fourth binding partner may be a detectable label, for example, a biotinylated phycoerythrin, or a detectably labeled entity, for example, a biotinylated cyanine dye. The blocking species is biotin or a biotin derivative. Fifth binding partners include labeled antibodies and labeled oligonucleotides. Since avidin and streptavidin are tetravalent, the resultant layer (about 0.2um thickness) of magnetic streptavidin particles, attached to the surface of the microparticles, still possesses exposed and available streptavidin binding sites for binding of one or more biotinylated entities to form a preformed detectably labeled magnetic complex. Selective binding of a target entity present in a specimen sample to complementary preformed labeled magnetic complex and labeling with another target-specific binder bearing a different label results in formation of a detectable and differentiable analytical magnetic complex for each target entity. The analytical complex can be enriched magnetically prior to analysis and analyzed by appropriate detection means using to provide quantitative or semi-quantitative information on the target entity present in the specimen sample. For simultaneous analysis of multiple target entities a cocktail or set of discrete labeled magnetic particles with affinities for the individual target entities is prepared and added to the specimen sample suspected of containing the target species.

### BRIEF DESCRIPTION OF THE FIGURE

FIG. 1 is a representation of the magnetically labeled particle bound to a target entity where the particle and the target entity are further labeled with a detectable label.

### DETAILED DESCRIPTION OF THE INVENTION

This invention utilizes the following components and reagents to provide compositions and components suitable for the disclosed applications:

a. A mixture of discrete microparticles defined as a subset or “cocktail” of non-magnetic microparticles, each one in the size range of 0.5 to 30 um, unlabeled or bearing one or more different detectable internal labels, operably linked to a first binding partner, exemplified by biotin or a biotin derivative of comparable affinity;

b. Second binding partner, bound operably to and forming a layer on the surface of discrete magnetic particles in the size range of 50 to 1000 nm, exhibiting affinity for first, wherein second binding partner is exemplified by an avidin species,

c. Third binding partner is exemplified as a target specific antibody or nucleic acid sequence operably linked to first binding partner, thereby forming an outer layer of target specific binder on the magnetic particle layer on discrete magnetic microparticles recognizing a complementary specific amino acid or nucleotide sequence on the target entity,

d. Biotinylated fourth binding partner, bearing a second label at different levels, with affinity for second binding partner,

e. Fifth binding partner, labeled with a third differentiable label, and recognizing available epitopes on target entities not occupied by third binding partner,

f. A reagent defined as a blocking agent with affinity for second binding partner for binding to residual binding sites on second binder exemplified by biotin or a biotin species.

g. A cocktail or subset of discrete preformed labeled target specific magnetic complexes, formed by the sequence of steps in preceding sections a.) to f.), for simultaneous isolation and analysis multiple target entities.

h. Magnetic separators capable of producing high magnetic gradients external to the specimen container, also known as HGMS magnetic separators, which are used for both so called magnetic incubations, defined as incubations performed inside the magnetic separator, and for magnetic enrichment and collection of one of more target entities captured by means of discrete detectably labeled complexes,

i. Means for detection or quantitation of one or more different target entities captured by a subset of discrete detectably labeled magnetic complexes, herein defined as optical means including but not limited to flow cytometry analyzers, laser scanners and visual or instrumental microscopic platforms

The novel compositions disclosed in this invention are functionally linked and schematically depicted as follows:

(Microparticle/fluorA-biotin)-(Sav-nanoparticle-Sav)-(biotin-fluorB)

(Sav)-(biotin-binderD)-(target entity)-(binderE-fluorC)

| | | | | | | |
|---|---|---|---|---|---|---|
| size range: | 0.5-30 | um | 0.05-1.0 | um | 0.05-50 | um |
| preferred: | 1-10 | um | 0.1-0.5 | um | 1-20 | um |
| most preferred: | 1-5 | um | 0.1-0.3 | um | 5-20 | um |

wherein the microparticle may be non-magnetic, unlabeled or bearing one or more detectable labels (A) at variable ratios, and the magnetic particle also bears: 1. a detectable biotinylated label (B) at different levels, 2. a binding partner (D) specific for an epitope on target entity, wherein the target entity is further detectable by 3. another differentially labeled (C) binding partner (E) specific for a different epitope on target entity.

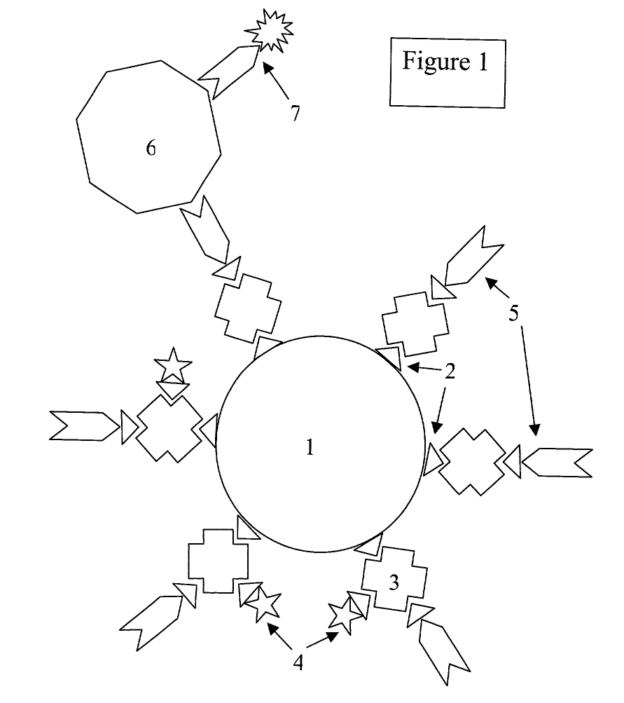

In FIG. 1, non-magnetic microparticles (1) operably linked with a first binding partner (2) capable of binding to a complementary second binding partner, which in turn, is operably linked to smaller magnetic particles (3). Free binding sites on the magnetic particles are available for binding additional entities: a third binding partner which is operably linked to first binding partner (5) and is specific for a target entity (6), or a detectably labeled fourth binding partner linked to the first binding partner (4). Additional detectable labels (7), which are target specific, and recognize available epitopes on bound target entities, provide further differentiation and discrimination of target entities.

### Microparticles

Bare microparticles of various sizes ranges also bearing one of more fluorescent dyes or the same dye at different levels are available from several vendors including Bangs Labs, Interfacial Dynamics, Molecular Probes, Polysciences, CPG. On the other hand, micro particles bearing biotin species are less readily available but can be prepared from the corresponding amino or carboxy functionalized microparticles by methods in the art. Incorporation of suitable fluorescent labels can be done by direct coating to achieve relatively weak surface labeling with different fluors as taught in the art. Or, preferably, by adding the dye or dye mixtures during the emulsion polymerization process in the preparation of the optically labeled microparticles bearing a single or mixed optical labels at varying ratios as disclosed by Richard et al. (U.S. Pat. No. 5,795,719). Processes for preparing magnetic polymeric latex particles are also known in the art (Daniel et al., U.S. Pat. No. 4,358,388). These processes if not the specific compositions of this invention are well known in the art.

### Magnetic Nanoparticles

As previously mentioned, the superparamagnetic particles used in this invention are disclosed in the art. The preferred particles consist of a magnetite core or cluster of smaller magnetite domains coated with a denatured layer of albumin to which streptavidin is covalently attached. The particle size range is 0.5 to 1 um, preferably 0.1 to 0.5um and most preferable 0.1 to 0.3 um, all of which can be efficiently collected in 15 ml or 50 ml plastic centrifuge tubes placed in an appropriate external HGMS magnet system. The magnetic collection times for separation are typically 5 to 20 minutes depending on the viscosity of the medium, but are shorter (about 5 minutes) for magnetic incubations. The streptavidin binding capacity for the most preferred particles is about 1 to 10 nmoles biotin/mg iron with substantially no non-specific binding of non-target entities present in complex specimen samples such as blood.

### Avidin Species

Streptavidin is the avidin species of choice for immobilization on the magnetic nanoparticles, since it has a lower isoelectric point than avidin, thereby exhibiting lower non-specific binding of non-target entities. Binding of biotin to both avidin and streptavidin is of extremely high affinity (10E+15 1/M) making dissociation of a biotinylated binding partners from avidin species with soluble biotin difficult to perform even under harsh conditions such as high or low pH or chaotropic ions.

The target specific binding partners are typically antibodies, primarily monoclonal but also polyclonal, oligonucleotides and lectins. Biotinylation of antibodies, olignucleotides and other binder entities is widely practiced in the art and well described in Bioconjugation (M. Aslam and A. Dent, Macmillan, 1998). A low level of biotin substitution on the antibody, generally averaging about one biotin per antibody, is preferred for attaching biotinylated antibodies to streptavidin-coated nanoparticles to minimize the occurrence of aggregates due to crosslinking by multiple biotins on the antibody.

### Magnetic Separators and Separations

Magnetic particles in the most preferred size range of 100 to 300 nm, bound to target entities, require relatively high magnetic gradients for efficient and rapid collection in a short time frame of 5 to 20 minutes. The preferred magnetic separators are the previously cited external HGMS systems, which generate a high gradient external to the vessel containing the specimen sample. The most preferred magnetic configurations are quadrupole or hexapole in design. Such HGMS can accommodate tubes up to 50 ml in volume.

The labeled magnetic complexes of this invention are preferably used as preformed labeled target-specific magnetic nanoparticle-microparticle complexes, also incorporating detectable labels, capable of binding to specific epitopes, defined as specific amino acid or nucleotide sequences, on target entities. For simultaneous analysis of multiple target entities, as disclosed in this invention, discrete sets of labeled magnetic microparticles complexes each specific for single target entity, are combined in subsets or cocktails.

Upon capture and separation of target entities from the specimen sample by magnetic means, the subset of magnetic particle complexes can be analyzed directly by appropriate means, including but not limited to optical means, for example, by flow cytometry, to provide information on the type and quantity of the individual target entities present in the specimen sample.

### Binding Partners

This preceding discussions focused on interactions of biotin and biotin analogs with avidin species as the binding partners, but are also applicable generically to numerous binding partner systems known in the art including but not limited to receptors, antibodies, antigens, lectins and oligonucleotides. Examples of biotinylated antibodies used in this invention are: HER2/neu, cytokeratins, mucin, all of which are applicable to the detection and diagnosis of circulating tumor antigens and cells.

For DNA/RNA assays, the biotinylated capture probes can be similarly bound to the streptavidin-magnetic particles in addition to the labeled detection binders. After capture of the target nucleic acid or oligonucleotide entity, a differentially labeled detection probe recognizing a nucleotide sequence on the target entity is used to identify the target entity.

The improvements and discoveries described and claimed herein provide greatly improved methodologies over prior art systems and methods, and to have applications including but not limited to microarray assays, nucleic acids assays, immunoassays and cell diagnostics. Thus, in accordance with the present invention, an improved diagnostic system is provided, which comprises:

a. Biotinylated microparticles bearing no detectable label

b. Biotinylated microparticles differentiable by bearing a single detectable marker at different intensity levels

c. Biotinylated microparticles differentiable by bearing more then one different detectable markers at variable relative ratios

d. Substantially monodisperse biotinylated microparticles differentiable by differences in mean particle diameters

e. Avidin or streptavidin coated magnetic particles of different sizes and magnetic characteristics

f. Magnetic particle-microparticles complexes with binding capacities for several biotinylated binding partners or biotinylated labeled species

g. Magnetic biotinylated particle-microparticle complexes each bearing a single target specific binding partner

h. A cocktail of different labeled and unlabeled magnetic microparticles each specific for a different target entity

i. Biotinylated labeled binding partners for labeling the magnetic particles

j. Differentially labeled binding partners with affinity for epitopes on the target entities

k. Biotinylated reagents for blocking residual binding sites on magnetic particles.

Thus, the invention utilizes microparticles and magnetic particles to form a magnetic microparticle which serves as the base material for various application enumerated below. The disclosed particles are particularly useful in microarrays for detection of multiple targets in the same specimen under conditions where conventional magnetic nanoparticles by themselves cannot be readily analyzed.

Further applications of the numerous combinations of the above-cited variables include different binding partners providing means for simultaneous isolation and analysis of multiple target entities from a single specimen sample by appropriate detection and sizing means including but not limited to flow cytometry, fluorimetry, laser scanning, microscopic imaging, particle sizing instruments.

It is a primary object of the present invention to provide improved methods for magnetic enrichment, isolation and quantitation of single target entities from complex mixtures. It is a further object of the present invention to provide novel methods for simultaneous magnetic enrichment, isolation and detection of a plurality of target entities from complex mixtures. A further objective of the present invention is to provide novel compositions enabling aforementioned applications.

It is to be understood and appreciated that these discoveries in accordance with the invention are only those that are illustrative of the many additional potential applications of the compositions and methods that may be envisioned by one of ordinary skill in the art, and thus are not in any way intended to be limiting of the scope of the invention. Accordingly, other objects and advantages of the invention will be apparent to those skilled in the art from the following detailed description, together with the appended claims.

The improvements provided by the present invention will be readily apparent to one skilled in the art by comparison with the above-described U.S. Patents and other known methodologies of the prior art, and the preferred embodiments described herein.

One particularly advantageous aspect of the present invention is that it provides novel compositions suitable particularly for analysis of target entities present at low concentrations and thus requiring enrichment prior to analysis.

The improvements provided by this invention enable its use for enrichment, isolation and analysis of a single specific target entity.

The improvements provided by the invention also enable its use for the simultaneous enrichment, isolation and simultaneous analysis of a plurality of specific target entities.

The improved compositions and methods of this invention can be employed to assist in clinical diagnosis.

It has further been found that the improved diagnostic system of the invention can be employed as a means for monitoring patient responses to therapy.

The improved compositions of this invention can further be employed in ultrasensitive assays for detecting or quantitating single or multiple soluble antigens including, but not limited to microarray assays, nucleic acid assays and immunoassays.

It will be apparent to those skilled in the art that the improved diagnostic systems provided by the invention can be utilized as a method for enrichment, isolation and analysis of target species including but limited to microarray assays, immunoassays, nucleic acid assays and cell diagnostics.

The present invention has been developed keeping in mind such potential users of the methods for this purpose.

The preferred embodiments of the invention, which incorporate these improvements, as described previously have also been found, unexpectedly, to enable the invention to be employed in many fields and applications additional to those enumerated.

The invention will now be described in terms of a particularly preferred example for isolating and analyzing single target cell species from blood using the following step sequence in Example 1.

### Example 1

1 Starting with commercially available biotin-microparticles of about 6 um diameter.

2 Adding the microparticles to an excess of streptavidin-coated magnetic nanoparticles of about 200 nm diameter to deposit a layer of streptavidin-nanoparticles on the microparticles.

3 Removing the excess streptavidin-nanoparticles from the streptavidin-coated microparticles by low-speed centrifugation.

4 Removing any microparticles, which are not magnetic from magnetic beads by magnetic separation. These microparticles will serve as base material and will be converted to three different microparticle sets named as M-1, M-2 and M-3.

5 M-1: Adding an optimized amount of anti-her2-neu antibody-biotin conjugate and phycoerythrin-biotin conjugate (PE-biotin) at 1× level. Her2neu antibody recognizes an her2neu antigen present in the serum specimen. PE-biotin dye conjugates bound at different levels to the magnetic microparticles differentiates M-1 from M-2 and M-3.

6 M-2: Adding an optimized amount of anti-cytokeratin antibody-biotin conjugate and biotin-PE at 3× level. Anti cytokeratin antibody recognizes keratin antigen present in the specimen. Thus M-2 will have a 3×higher PE level than M-1.

7 M-3: Adding an optimized amount of anti-mucin antibody-biotin conjugate and biotin-PE at a 10×level. Anti mucin antibody recognizes mucin antigen present in the specimen. M-3 will have a 10×higher PE level than M-1 to differentiate these three microparticle subsets from each other.

8 Blocking the remaining unused sites with a slight excess of biotin or biotinylated albumin and purifying by low-speed centrifugation.

9 Combining the three subsets to form a single reagent cocktail.

10 Adding this cocktail and three different APC (allophycocyanin) labeled antibodies consisting of anti-her2neu-APC, anti-cytokeratin-APC and anti-mucin-APC) to a specimen sample wherein the APC-antibodies and the PE-antibodies on the microparticles recognize different epitopes on the target entities.

11 Incubating for two 10 min magnetic incubation cycles inside a magnetic separator to selectively bind the targets.

12 Separating the magnetic complexes including targets bound to complexes and excess magnetic complexes in the magnetic separator for about 15 minutes.

13 Washing the magnetic complexes magnetically to remove non-target materials.

14 Reconstituting the washed magnetic complex in about 1 ml buffer.

15 Analyzing the particle populations in a flow cytometer, CellTrack™ or another detector, which can separate events based on size and fluorescence intensity.

The three types of microparticle complexes will be separated based on PE staining intensity and for APC staining. If the sample contains only her2neu antigen, then only M-1 will be positive with APC corresponding to a certain intensity level based on the concentration of the target entity. If the sample contains all three target entities, then all three microparticles will be positive for APC dye and can be differentiated.

### Example 2

A similar protocol is used for simultaneous analysis of more than one target gene product. We can prepare different microparticles with different oligomeric probes. Each probe will be conjugated to a different level with PE dye. Different microparticles with different probes can thus be differentiated based on PE fluorescence intensity. Each oligomeric probe recognizes a specific gene marker, which is prelabeled with a second fluorescent dye. Thus several gene products can be detected simultaneously in the same specimen sample.

### Example 3

### Fluorescent Microbeads Prelabeled at Different Levels

This example provides a method for preparing microparticles with different levels of fluorescent dyes similar to Example 1, but using a different procedure. In this example, prelabeled fluorescent microbeads are used:

1 Starting with commercially available fluor-labeled biotin-microparticles of about 6 um diameter.

2 Adding the microparticles to an excess of streptavidin-coated magnetic nanoparticles of about 200 nm diameter to deposit a layer of streptavidin-nanoparticles on the microparticles.

3 Removing the excess streptavidin-nanoparticles from the streptavidin-coated microparticles by low-speed centrifugation.

4 Removing non-magnetic microparticles by magnetic means.

5. Adding an optimized amount of biotinylated antibody or an oligomeric probe having an affinity for a specific target.

6. Blocking the remaining unused sites on the streptavidin-nanoparticles with a slight excess of biotin or biotinylated albumin and purifying by low-speed centrifugation.

It will be apparent to those skilled in the art that the improved diagnostic system of the invention is not to be limited by the foregoing description of preferred embodiments, e.g. involving only biotin and avidin species, but also includes cognate interactions of other binding partner combinations known in the art, and that any such limitations are only to be defined by the appended claims.

### Example 4

### Use of Fluorescent Magnetic Beads of the Present Invention as Calibration Beads

The novel compositions and methods provided by this invention, in particular the use of labeled magnetic particles in conjunction with fluorescent dyes, can be used as calibrators for instrument systems, such as in the CellSpotter® and CellTracks™ (U.S. Pat. Nos. 5,985,153 and 6,136,128) instrument systems.

Fluorescent dyes are commonly used to detect specific targets (cells) or analytes by means of different analytical platforms such as flow cytometers and microscopes. These instruments are equipped with different light sources to excite fluorescent dyes and with photomultiplier tubes (PMT's) or other detectors to detect the emitted signals. The outputs of the light sources and the detectors may become variable with time, which will influence both signal generation and detection. Hence, the level of signal detection with targets may change with time. It is necessary to periodically or continuously adjust the light source and the detectors to equalize the responses for a given number of fluorescent molecules, e.g. by appropriate external calibrators.

Beads or microparticles bearing different fluorescent dyes are available commercially for calibration of flow cytometers and fluorescence microscopes. The microparticles, also known as latex particles, are made of polystyrene that are doped with or coupled to a single or a plurality of fluorescence dyes. These calibration beads are used routinely to adjust detector outputs and signal to noise ratios in flow cytometers. The disadvantage with these beads is that they are not magnetic and cannot be used in analytical procedures requiring magnetic beads in analytical platforms. These analytical platforms detect targets that are magnetic and fluorescent. The targets can be made magnetic by labeling targets with magnetic particles conjugated to antibodies specific for targets. The novel beads disclosed in this invention are labeled with both magnetic particles and fluorescent dyes that can be used in the CellSpotter® and CellTracks™ systems as calibration beads.

There are several methods for preparing magnetic calibration beads. One could attach small magnetic particles to commercial calibration beads by absorption or by using conjugation chemistries to couple magnetic particles to fluorescent beads. The sizes of fluorescent beads could range from 1-20um. The magnetic particles for conjugation are preferably less than 0.2um. In the preferred mode, commercially available 6um non-magnetic red beads (Deep red beads from Molecular Probes, Eugene, Oreg., Part Number L-14819) were made magnetic by direct absorption of protein-coated Immunicon ferrofluids as follows:

Deep red beads (5×10⁶ beads/ml) were washed with excess phosphate buffered saline (PBS) by centrifigation to remove any detergent present in the bead sample. Bovine Serum Albumin (BSA) coated magnetic particles (Immunicon, Part Number 6020) were absorbed onto Deep red beads by mixing the washed beads and the magnetic particles at room temperature for 3 hours. The unbound magnetic particles were removed by centrifugation at 300×g where free magnetic particles will stay in the supernatant due to their small size. The magnetic beads were then resuspended in PBS and washed in a magnetic separator (HGMS; Immunicon QMS) to remove beads that were non-magnetic. The beads were then resuspended in PBS. The amino groups on the outer surface of the protein-coated magnetic particles were then crosslinked with excess 0.5% paraformaldehyde (PFA) for 2 hours at room temperature to improve stability of the coated beads. The beads were washed again by centrifugation to remove excess PFA. The beads were then resuspended and stored in PBS with BSA prior to use (PFA also introduces free aldehyde groups which can be utilized for attaching additional protein layers or quenched with a substance containing amino groups, for example, as shown above, with BSA, to minimize bead aggregation). The coating procedure did not alter the physical or fluorescence properties of the beads and thus allowed their use as calibration beads in the CellSpotter and CellTracks instruments.

What is claimed is:

1. A composition for the detection of one or more target entities in a mixture, said composition comprising non-magnetic microparticles or beads operably linked with a first binding partner capable of binding to a complementary second binding partner, said second binding partner being operably linked to magnetic particles smaller than the average size of said non-magnetic microparticles, thereby to form a layer of magnetic particles on said non-magnetic microparticles, wherein said magnetic particles bear variable amounts of free binding sites distal to the surface of said non-magnetic microparticles and wherein the bound magnetic particles are capable of binding both a labeled first binding partner and said first binding partner either in its free form or operably linked to one or more entities including entities for capturing said target entities in the mixture.

2. The composition of claim 1 wherein said non-magnetic microparticles are unlabeled.

3. The composition of claim 1 wherein said non-magnetic microparticles are detectably labeled.

4. The composition of claim 1 wherein said first binding partner is a biotin species.

5. The composition of claim 1 wherein said complementary second binding partner is an avidin species.

6. The composition of claim 1, wherein said free binding sites on said layer of magnetic particles are capable of directly or indirectly binding at least three additional entities.

7. The composition of claim 6 wherein said additional entities are selected from the group consisting of: a) one or more target specific third binding partners operably linked to said first binding partner; b) one or more detectable labels linked to first binding partner, c) a biotin species with affinity for blocking residual binding sites on the second binding partner located on the magnetic microparticles; and d) one or more detectably labeled target specific fourth binding partners which are capable of binding one or more epitopes on bound target entities.

8. The composition of claim 7 wherein said third binding partner is an oligonucleotide probe operably linked to said first binding partner and wherein said probe is complementary to a target oligonucleotide sequence.

9. The composition of claim 7 wherein said third binding partner is an antibody.

10. The composition of claim 7 wherein said fourth binding partner is a target specific binder capable of recognizing epitopes on target entities different from those recognized by said third binding partner.

11. The composition of claim 7, wherein the detectable label is a fluorescent compound.

12. A method for the detection of one or more target entities in a mixture, which method comprises the steps of adding the composition of claim 1 to the mixture, incubating the mixture, separating the components of the mixture by magnetic means, and detecting one or more of the target entities in the mixture.

13. The method of claim 12, wherein said entities are selected from the group of particulate analytes and non-particulate analytes.

14. A kit for the detection of one or more target entities in a mixture, which kit comprises the composition of claim 1, an incubation means, a magnetic separation means, and a detection means.

15. A composition useful in the calibration of a diagnostic instrument system which utilizes fluorescent detection, said composition comprising magnetic particles bound to particles having a detectable fluorescent label, said magnetic particles having an average size less than that of the particles having a detectable fluorescent label.

16. The composition of claim 15, wherein the magnetic particles have an average size of less than 0.2 um.

17. The composition of claim 15, wherein the particles having a detectable fluorescent label have an average size of from about 1 to about 20 um.
